(19) United States
(12) Patent Application Publication
(10) Pub. No.: US 2012/0029564 A1
(43) Pub. Date: Feb. 2, 2012

(54) Composite Rod for Spinal Implant Systems With Higher Modulus Core and Lower Modulus Polymeric Sleeve

(75) Inventors: Hai H. Trieu, Cordova, TN (US); Julien J. Prevost, Memphis, TN (US)

(73) Assignee: WARSAW ORTHOPEDIC, INC., Warsaw, IN (US)

(21) Appl. No.: 12/846,196

(22) Filed: Jul. 29, 2010

(51) Int. Cl. A61B 17/70; B23P 11/02

(52) U.S. Cl. 606/246; 29/447

(57) ABSTRACT

A spinal rod includes a metal component and a tube. The core component has a radius. The tube has a first state with a first state tube inner radius and a first state tube outer radius. The first state tube inner radius is greater than the core component radius. The tube has a second state with a second state tube inner radius and a second state tube outer radius. The second state tube inner radius is generally equal to the core component radius. The tube is deformable from the first state to the second state.

### FIELD OF INVENTION

Embodiments of the invention relate to spinal fixation systems having at least one composite component. More particularly, the embodiments relate to rods for use in spinal fixation systems that are composites of polyetheretherketone (PEEK) and metals or metal alloys.

### BACKGROUND

The spinal column is a biomechanical structure composed primarily of support structures including vertebrae and intervertebral discs and soft tissue structures for motive and stabilizing forces including muscles and ligaments. The biomechanical functions of the spinal column include support, spinal cord protection, and motion control between the head, trunk, arms, pelvis, and legs. These biomechanical functions may require oppositely designed structures. For example, the support function may be best addressed with rigid load bearing structures while motion control may be best suited for structures that are easily movable relative to each other. The trade-offs between these biomechanical functions may be seen within the structures that make up the spinal column. Damage to one or more components of the spinal column, such as an intervertebral disc, may result from disease or trauma and cause instability of the spinal column and damage multiple biomechanical functions of the spinal column. To prevent further damage and overcome some of the symptoms resulting from a damaged spinal column, a spinal fixation device may be installed to stabilize the spinal column.

A spinal fixation device generally consists of stabilizing elements, such as rods or plates, attached by anchors to the vertebrae in the section of the vertebral column that is to be stabilized. The spinal fixation device restricts the movement of the vertebrae relative to one another and supports at least a part of the stresses created by the weight of the body otherwise imparted to the vertebral column. Typically, the stabilizing element is rigid and inflexible and is used in conjunction with an intervertebral fusion device to promote fusion between adjacent vertebral bodies. There are some disadvantages associated with the use of rigid spinal fixation devices, including decreased mobility, stress shielding (i.e. too little stress on some bones, leading to a decrease in bone density), and stress localization (i.e. too much stress on some bones, leading to fracture and other damage).

In response, flexible spinal fixation devices have been employed. These devices are designed to support at least a portion of the stresses imparted to the vertebral column but also allow a degree of movement. In this way, flexible spinal fixation devices avoid some of the disadvantages of rigid spinal fixation devices. These devices may be made of a material having a lower modulus of elasticity, or by combining materials in complex manufacturing processes to create composites having more flexibility.

The description herein of problems and disadvantages of known apparatuses, methods, and devices is not intended to limit the invention to the exclusion of these known entities. Indeed, embodiments of the invention may include, as a part of the embodiment, portions or all of one or more of the known apparatus, methods, and devices without suffering from the disadvantages and problems noted herein.

### SUMMARY OF THE INVENTION

An embodiment of the invention may include a spinal rod having a core component and a tube. The core component has a radius. The tube has a first state with a first state tube inner radius and a first state tube outer radius. The first state tube inner radius is greater than the core component radius. The tube has a second state with a second state tube inner radius and a second state tube outer radius. The second state tube inner radius is generally equal to the core component radius. The tube is deformable from the first state to the second state. The core component and the tube together have a modulus of elasticity at least 10% less than the modulus of elasticity of the metal component.

Another embodiment of the invention may include a method of forming a composite rod. A step may include nesting a plurality of tubes over a core. Another step applies a deforming force to the plurality of tubes such that each tube places a hoop stress on the metal component.

Yet another embodiment may include a spinal rod comprising a core component and a plurality of nested tubes. The core component has an outer surface. Each tube of the plurality of nested tubes may have a first state wherein the tube is dimensioned larger than the outer surface of the core component and formed to enclose the outer surface of the core component. Each tube may also have a second state dimensioned smaller than the first state such that when the plurality of tubes undergo a deformation from the first state to the second state each tube places a hoop stress on the core component.

Additional aspects and features of the present disclosure will be apparent from the detailed description and claims as set forth below.

### BRIEF DESCRIPTION OF THE DRAWINGS

FIG. 1 is a view of a cross section of a spinal rod according to an embodiment of the present invention.

FIG. 2 is a view of a cross section of a spinal rod according to another embodiment of the present invention.

FIG. 3 is an exploded view of parts of a spinal rod according to the embodiment of FIG. 1.

FIG. 4 is a partial cutaway side view of parts of a spinal rod according to the embodiment of FIG. 1 prior to a heating process.

FIG. 5 is the partial side view of FIG. 4 after a heating process forms a composite spinal rod.

FIG. 6 is a partial exploded view of parts of a spinal rod according to the embodiment of FIG. 2 prior to a heating process.

FIG. 7 is a partial side view of an embodiment of a spinal rod before a second heating process forms a composite spinal rod.

FIG. 8 is the partial side view of FIG. 7 after a second heating process forms a composite spinal rod.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

It is a feature of an embodiment of the present invention to provide composite rods for use in spinal fixation systems. The composite components may comprise a first core material which may be a metal, metal alloy, a polymer, or a polymeric composite; and a second material formed in a sleeve and selected from the group consisting of resorbable and non-resorbable polymeric materials. In a preferred embodiment, the composite comprises polyetheretherketone tube or sleeve and a metal or metal alloy core.

Another feature of an embodiment of the present invention may provide a core material having a higher melt temperature and a higher modulus of elasticity than a sleeve or tube member. The higher core melt temperature allows for the core to remain intact while the tube or sleeve is deformed around the core.

Polyetheretherketone (PEEK) is a polymer that is commercially available from a number of suppliers and also is available in medical grades that are preferred for use in the embodiments (e.g., PEEK OPTIMA™, commercially available from Invibio Ltd., Lancashire, United Kingdom). The resorbable and non-resorbable polymeric materials, such as PEEK, can be combined with at least one metal or metal alloy in accordance with the embodiments in order to form composite components such as rods and plates for use in spinal fixation systems. Preferred metal and metal alloys for use in the invention include, but are not limited to, titanium, titanium alloys (e.g. Ti-6Al-4V), tantalum, tantalum alloys, stainless steel alloys, cobalt-based alloys, cobalt-chromium alloys, cobalt-chromium-molybdenum alloys, niobium alloys, nickel-titanium alloys (Nitinol), and zirconium alloys.

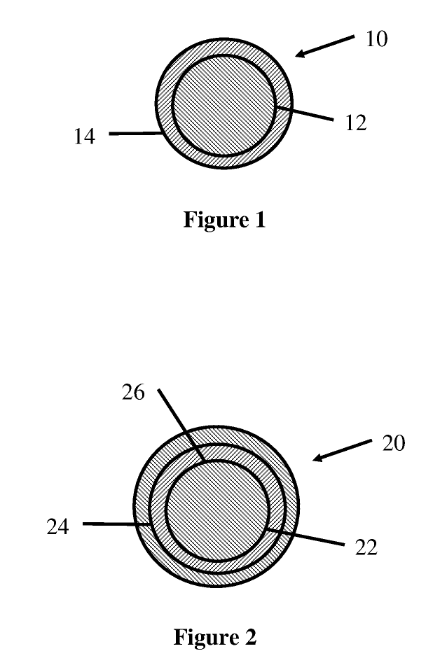

Turning now to FIG. 1, FIG. 1 is a view of a cross section of a spinal rod 10 according to an embodiment of the present invention. The cross section of the spinal rod 10 comprises a central rod or inner core of metal 12 and an outer sleeve or tube of PEEK 14. The diameters of the inner metal core 12 and outer polymer tube 14 may be adjusted to change the modulus of elasticity of the composite. The modulus of elasticity of the construct, though, is bounded by the lower limit of the polymer and the upper limit of the metal. As the radius of the metal core 12 approaches the total construct radius, the modulus of elasticity of the construct approaches the modulus of the metal core 12. Similarly, as the thickness of the polymer tube 14 approaches the total construct radius, the modulus of elasticity of the construct approaches the modulus of the polymer tube 14. This allows, then, a construct having a specific radius with a modulus of elasticity that may vary based upon the size of the individual components.

Turning now to FIG. 2, FIG. 2 is a view of a cross section of a spinal rod 20 according to another embodiment of the present invention. The cross section of the spinal rod 20 comprises a central rod or inner core of metal 22, a first outer sleeve or tube of PEEK 24 and a second outer sleeve or tube of PEEK 26. The diameters of the inner metal core 22 and outer polymer tubes 24 and 26 may be adjusted to change the modulus of elasticity of the composite. The modulus of elasticity of the construct, though, is bounded by the lower limit of the polymers and the upper limit of the metal. As the radius of the metal core 22 approaches the total construct radius, the modulus of elasticity of the construct approaches the modulus of the metal core 22. Similarly, as the thickness of the polymer tube 24 or 26 approaches the total construct radius, the modulus of elasticity of the construct approaches the modulus of the lower modulus of the polymer tubes 24 or 26.

The polymer tubes 24 and 26 may be of different moduli of elasticity. It may be beneficial to use multiple tubes 24 and 26 as the total thickness of the polymer tubes 24 and 26 increases. As a deforming force (such as the forces derived from the application of heat) is applied to the polymer tubes 24 and 26, the tubes may shrink to bond with the inner metal core (for the inner polymer tube 24) or bond with the inner polymer tube 24 (for the inner polymer tube 26). The amount of heat may be reduced by having multiple tubes as the amount of heat required to shrink the tube is a function of the tube thickness. Thus, having multiple tubes put on serially between heating processes may require less heat than a single, thicker tube. While heat shrinking is the preferred method for shrinking the tube 26 to the bonded configuration, other methods such as chemical methods may be used to deform the tube from the first configuration to the second configuration.

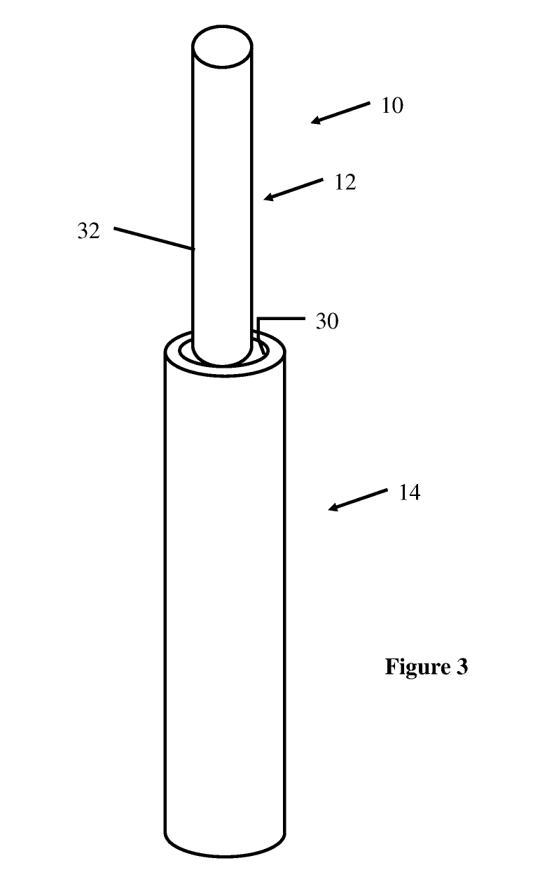

Additionally, thinner tubes or sleeves may be easier to advance over the inner metal core. Thinner tubes or sleeves may be generally more flexible to bending along the length of the tube, and thus may be able to advance over the inner core more readily than thicker tubes of the same material. In addition to the thickness, the relative radii of the tube and the inner core also affect the ease of advancement of the tube over the core. There is a tradeoff between ease of advancement over the inner core and inner diameter of the inner tube. The inner diameter of the inner polymer sleeve or tube may be larger than the outer diameter of the inner metal core. As that difference in diameter gets larger, the tube is more easily advanced over the inner core. However, as the difference becomes greater, the amount of shrinking required to bond the polymer to the inner core would be greater. As is shown in FIG. 3, FIG. 3 is an exploded view of parts of a spinal rod 10 according to the embodiment of FIG. 1. The inner metal core 12 and the outer polymer tube 14 are sized to allow the inner core 12 to slide within the polymer sleeve 14. An inner wall 30 of the sleeve 14 has a radius greater than the radius of an outer wall 32 of the inner core 12. Thus, the inner core 12 may be received within the tube 14 without interference between the two wall surfaces 30 and 32.

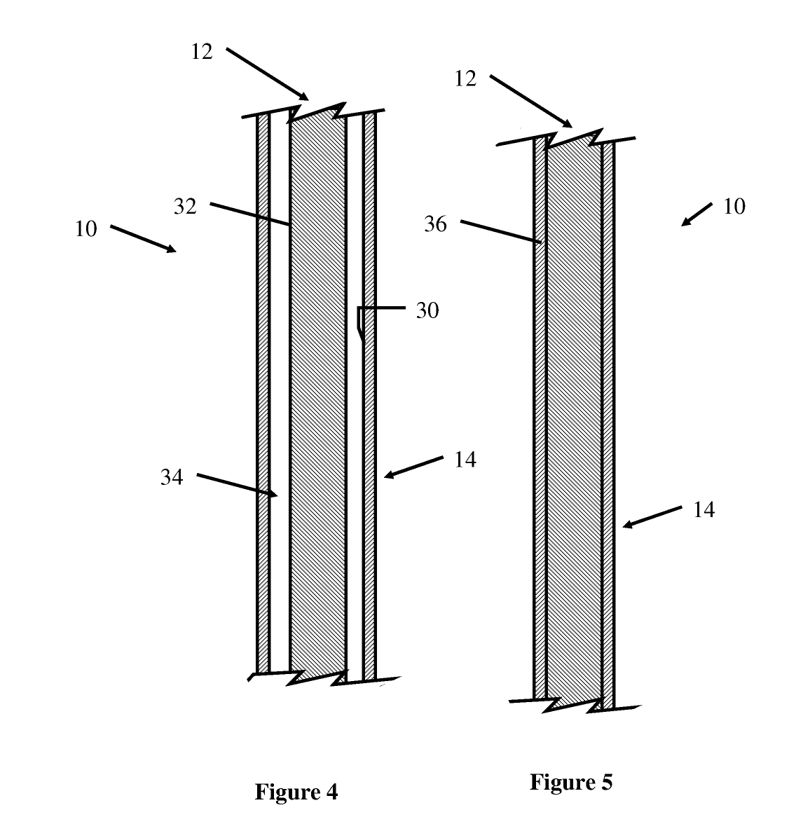

Turning now to FIGS. 4 and 5, FIG. 4 is a partial cutaway axial view of parts of a spinal rod 10 according to the embodiment of FIG. 1. FIG. 5 is the partial axial view of FIG. 4 after a heating process forms a composite spinal rod 10. A void 34 between the inner core wall 30 and the polymer sleeve wall 32 allows the inner core 12 to advance within the tube 14. When the outer tube 14 is heated, then the tube 14 may shrink to bond to the surface 30 of the inner core 12 at a bonding surface 36.

The diameters of the inner metal core 12 and outer polymer tube 14 may be adjusted to change the modulus of elasticity of the composite. The modulus of elasticity of the construct, though, is bounded by the lower limit of the polymer and the upper limit of the metal. As the radius of the metal core 12 approaches the total construct radius, the modulus of elasticity of the construct approaches the modulus of the metal core 12. Similarly, as the thickness of the polymer tube 14 approaches the total construct radius, the modulus of elasticity of the construct approaches the modulus of the polymer tube 14. This allows, then, a construct having a specific radius with a modulus of elasticity that may vary based upon the size of the individual components. As previously described, thinner tubes may be easier to advance over the inner metal core. There is a tradeoff between ease of advancement over the inner core and inner diameter of the inner tube. The inner diameter of the inner polymer sleeve or tube may be larger than the outer diameter of the inner metal core. As that difference in diameter gets larger, the tube is more easily advanced over the inner core. However, as the difference becomes greater, the amount of shrinking required to bond the polymer to the inner core would be greater.

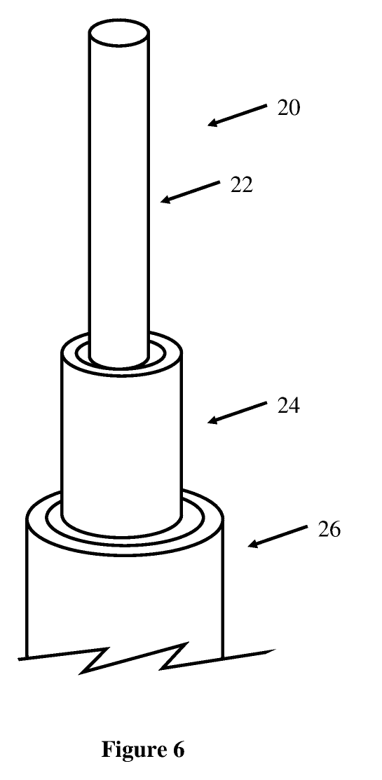

Turning now to FIGS. 6 through 8, FIGS. 6 through 8 correspond to an embodiment similar to the embodiment shown in FIG. 2. FIG. 6 is a partial exploded view of parts of a spinal rod 20 according to the embodiment of FIG. 2. The cross section of the spinal rod 20 comprises a central rod or inner core of metal 22, a first outer sleeve or tube of PEEK 24 and a second outer sleeve or tube of PEEK 26. The diameters of the inner metal core 22 and outer polymer tubes 24 and 26 may be adjusted to change the modulus of elasticity of the composite. The modulus of elasticity of the construct, though, is bounded by the lower limit of the polymers and the upper limit of the metal. As the radius of the metal core 22 approaches the total construct radius, the modulus of elasticity of the construct approaches the modulus of the metal core 22. Similarly, as the thickness of the polymer tube 24 or 26 approaches the total construct radius, the modulus of elasticity of the construct approaches the modulus of the lower modulus of the polymer tubes 24 or 26.

As previously described, the relative radii of the parts may be sized to allow for ease of advancement of the parts coaxial to one another. The outer tube 26, however, may be sized based on the shrunken inner tube 24 radius or the pre-heat treated diameter of the inner tube 24. Heat applied to the tubes to shrink the tubes 24 and 26 to the bonded state may be applied serially (thus allowing the smaller outer tube 26 diameter) or may be applied in parallel thereby requiring the larger inner diameter for the outer sleeve 26. The outer sleeve 26, if heated in parallel or in a composite where the outer sleeve is advanced unto the composite prior to the inner tube 24 being shrunken, must shrink more than in a composite where the outer tube 26 is not advanced over the inner tube 24 until after the inner tube 24 is bonded to the inner core 22.

While the embodiments have shown one or two tubes in use, in practice, as many tubes as desired for a final thickness may be used. The tubes may have the same modulus of elasticity as other tubes, or may have differing moduli of elasticity depending on the need. As described above, thinner tubes may be easier to advance over the inner metal core as a tradeoff between ease of advancement over the inner core and inner diameter of the inner tube. As that difference in diameter gets larger, the tube is more easily advanced over the inner core. However, as the difference becomes greater, the amount of shrinking required to bond the polymer to the inner core would be greater. Thus, multiple, thinner tubes may be beneficial instead of thicker tubes.

Additionally, the tubes may vary in length and thickness from each other in order to allow for a composite rod having varying thickness along the length of the rod. The thickness of the tubes may be between 0.1 mm and 1 mm, and preferably between 0.25 mm and 0.75 mm. For example, if one end of the rod needs to be thicker, then sleeves having lengths shorter than the length of the core may be used at the end that is desired to be thicker. The additional layers at this end may make the implant thicker at that end, and thus achieve variable thickness along the length of the rod.

When the tubes are heated over the core, the tubes shrink to the core. The shrinking press fits the tube to the outside of the core, applying a hoops stress to the core. A friction fit between the tube and the core is achieved, forming the composite rod. The tube thickness, during this process, becomes slightly larger than the thickness of the tube before it is shrunk to the core size. However, the outer radius of the tube is still less than its original outer radius as the inner diameter of the tube has reduced its size more than the thickness has changed.

Other processes may help to hold the tubes over the core. For example, adhesives may be added between the tube and the core to allow for additional pull out strength between the core and the tube. Surface texturing (such as surface roughening) may also increase the pull out strength between the core and the tube. Similarly, surface structures (such as grooves) may be cut into the core surface to increase the pull out strength.

One use of rods made according to this invention may be in revision cases. In these types of spinal implant systems, the screws inserted into the vertebra have a rod-capturing portion that is sized according to the original rod diameter. The original rod may need to be a more rigid construct immediately after surgery. Thus, a solid metal (and thus high modulus of elasticity) material may be used. As healing progresses and the vertebra fuse together more completely, the spinal implant system may not need to be as rigid. However, given the other hardware already implanted (namely the rod-capturing portion of the spinal implant system), a similarly sized rod would be the most effective rod to replace within the system. The rod shown above may provide a rod having the same size as the original rod in the system while allowing for a lower modulus of elasticity.

The deformation process is preferably a heating process. Because the inner core is made of metal, it has a relatively high melting temperature. For example, titanium has a melting temperature of 1670° C. Stainless steel melts at 1510° C., while titanium Ti-6Al-4V melts at above 1600° C. The tubes, made of a PEEK material melt at around 340° C. The process is not meant to completely melt the PEEK material. The shrink process, then, may occur at low temperatures relative to the melting temperature of the metal component, and may occur even at temperatures below the melting temperature of the tube material. As the material approaches melting, it begins to deform and shrink in inner diameter size. The tube may continue to shrink until it abuts the inner core. The inner core resists additional shrinking of the tube. The tube then stresses the outside of the core, thereby applying a hoops stress to the core. This stress creates the friction fit between the tube and the core. Additionally, an adhesive may be used inside the tube to increase the contact forces between the tube and the core. For example, a thermoplastic adhesive may be used.

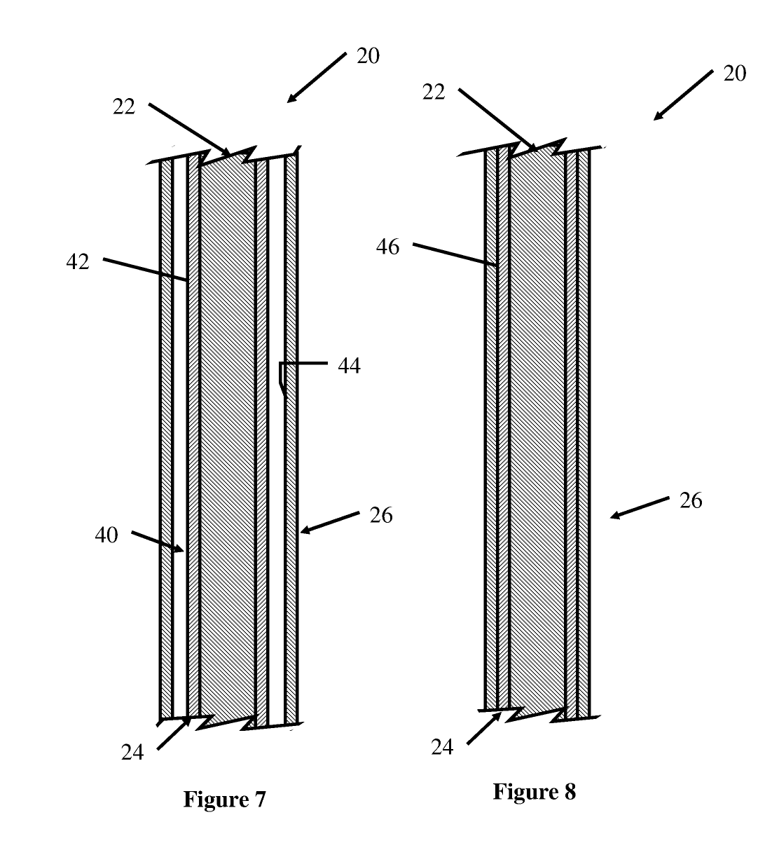

FIG. 7 is a partial axial view of an embodiment of a spinal rod 20 before a second heating process forms a composite spinal rod 20 while FIG. 8 is the partial axial view of FIG. 7 after a second heating process forms a composite spinal rod 20. The composite inner portion of FIGS. 7 and 8 (core 22 and inner tube 24) may be formed as previously described with respect to FIG. 1. The first heating process has already bonded the inner tube 24 to the metal core 22. A void 40 between the inner tube 24 and the outer tube 26 allows the outer tube 26 to advance over the inner tube 24. When the outer tube 26 is heated, then the tube 26 may shrink to bond an outer surface 42 of the inner tube 24 to an inner surface 44 of the outer tube 26 at a bonding surface 46.

It should be apparent that the composite components provided by the embodiments may take a myriad of different forms or configurations, in accordance with the guidelines provided herein. Therefore, one of skill in the art will appreciate still other configurations for composite spinal fixation components in accordance with the embodiments. For example, the metal and polymer portions of each composite component may have varying thicknesses and geometries, and need not correspond to the relatively uniform thicknesses and geometries depicted in the figures. Additionally, as the different forms change from generally round configurations, the meaning of “diameter” and “radius” must accordingly adjust from a strict interpretation requiring a circular cross section to allow for the structures of other shapes to fit within these aspects of the invention. Namely, the definitions should submit to an interpretation where an inner core has a centroid and the distance at all polar orientations around that centroid to the inner diameter of the hollow cylindrical tube or sleeve member is greater than the distance to the outer boundary of the inner core before the process to shrink the outer tube has begun. In other words, the shape of the tube should be slidably received over the shape of the core. Accordingly, skilled artisan will appreciate that an infinite number of variations in cross sections of the composite rods provided for by the embodiments may occur, in accordance with the guidelines provided herein.

Although FIGS. 1-8 were illustrated with respect to PEEK/metal composites, according to embodiments of the invention other resorbable and non-resorbable polymeric materials may be used in place of PEEK in the composite structures. For example, a resorbable polymer material such as polylactides (PLA), polyglycolides (PGA), copolymers of (PLA and PGA), polyorthoesters, tyrosine, polycarbonates, and mixtures and combinations thereof may be used in lieu of PEEK. Also, non-resorbable polymeric material such as members of the polyaryletherketone family, polyurethanes, silicone polyurethanes, polyimides, polyetherimides, polysulfones, polyethersulfones, polyaramids, polyphenylene sulfides, and mixtures and combinations thereof alternatively may be used in lieu of PEEK. Therefore, a wide variety of composite components may be fabricated in accordance with the embodiments.

PEEK generally has a lower modulus of elasticity and tensile strength than the exemplary metals and metal alloys shown in the table. The differences in physical properties between PEEK and the metals can be advantageously utilized in the embodiments by fabricating the composite spinal fixation rods with appropriate proportions of PEEK and metal, metal alloy, or mixtures thereof to produce a device having the desired physical properties. In this way, composite components can be fabricated having, for example, an average or mean modulus of elasticity different from that of the modulus of elasticity of any of its individual components. For example, consider two rods with the same diameter—the first rod of Ti-6Al-4V and the second rod a composite of Ti-6Al-4V and PEEK. Because a portion of the second rod comprises a material having a lower modulus of elasticity (PEEK), than the modulus of elasticity of Ti-6Al-4V, the second rod will have a lower average or mean modulus of elasticity than the first rod. In general, a composite rod will have average or mean properties, such as average or mean modulus of elasticity, proportionate to the ratio of the components that comprise the rod. One who is skilled in the art will appreciate how to select an appropriate ratio and orientation of the components that make up the systems, rods, plates, and other components based on the desired physical properties, in accordance with the guidelines described herein. For example, other polymeric materials such as those provided herein may be chosen for use in the composite components instead of PEEK, in order to produce composite components having different average or mean properties.

Fabricating composite components of spinal fixation systems may be advantageous because of the ability to produce composite components with average or mean properties not otherwise possible. For example, if a rod of a certain diameter is required for use with a given spinal fixation system, fabricating a composite rod having the required diameter using PEEK and metal composites may yield a composite rod with an average or mean modulus of elasticity not otherwise achievable for the required diameter rod, if fabricated from a non-composite material. Therefore, one advantage provided by the embodiments is that a spinal fixation system component may be fabricated having a different average or mean modulus of elasticity without changing the dimensions or geometry of the component. This may be highly advantageous, for example, where fixation systems are desired to be retrofitted or otherwise customized for use with patients that require a more flexible fixation system, but require components that imitate the dimensions and geometries of the original, non-composite components of the fixation systems. To aid these patients, composite components may be fabricated in accordance with embodiments herein.

In a preferred embodiment, composite spinal fixation rods may be fabricated that have physical properties not otherwise attainable in rods and plates that are composed purely of metals and metal alloys. Preferably, the composite rods and plates have a mean or average modulus of elasticity less than about 75 GPa. Additionally, it is preferable that the composite rods and plates have a mean or average tensile strength less than about 150 MPa. One skilled in the art will be capable of fabricating composite materials comprising PEEK and at least one metal or metal alloy that have one or more of these preferred physical properties.

In another preferred embodiment, composite spinal fixation components may be fabricated comprising PEEK and a metal or metal alloy having a mean or average modulus of elasticity from about 1.2 GPa to about 192 GPa. More preferably, components may be fabricated having a mean or average modulus of elasticity from about 2 GPa to about 100 GPa. Even more preferably, components may be fabricated having a mean or average modulus of elasticity from about 3 GPa to about 50 GPa.

For example, a titanium spinal rod has a modulus of elasticity of about 116 GPa. PEEK has a modulus of elasticity of around 3.6 GPa. For a similarly sized composite rod made of titanium and PEEK, the modulus of elasticity of the composite rod may be reduced by increasing the thickness of the tubes while decreasing the diameter of the metal core. The modulus of elasticity, though, is bounded by the PEEK modulus on the low end and the titanium modulus on the high end. Other material, though, may be used having different moduli, and thus different bounds for the composite modulus of elasticity. For example, a PEEK core may be used with a polyethylene tube to get a much lower average modulus of elasticity. The core material has a higher modulus of elasticity than the tube, and the melt temperature of the core is higher than the melt temperature of the tube.

Previous composite spinal fixation rods have been formed by utilizing a metal injection molding (MIM) technique to fabricate the metallic portion, and an injection molding technique to fabricate the non-metallic, or polymeric portion. Disadvantages of the MIM process include requiring application of several hundred tons of pressure to a mold. This results in high tooling costs and precision processes.

In another embodiment, the second material may be mixed or combined with a first material comprising a metal or metal alloy. Thus, each component may be a composite comprising the first material and the second material which may be used to fabricate various composite rods as has been described herein in regards to PEEK. The composites comprising a first material and second material as described herein may be advantageously used to fabricate spinal fixation system components having average or mean properties not otherwise attainable for a given dimension or size when using non-composite materials to fabricate the components.

The foregoing detailed description is provided to describe the invention in detail, and is not intended to limit the invention. Those skilled in the art will appreciate that various modifications may be made to the invention without departing significantly from the spirit and scope thereof.

Furthermore, as used herein, the terms components and modules may be interchanged. It is understood that all spatial references, such as “first,” “second,” “exterior,” “interior,” “superior,” “inferior,” “anterior,” “posterior,” “central,” “annular,” “outer,” and “inner,” are for illustrative purposes only and can be varied within the scope of the disclosure.

1. A spinal rod comprising:
a core component having an outer surface; and
a plurality of nested tubes each tube having a first state wherein the tube is dimensioned larger than the outer surface of the core component and formed to enclose the outer surface of the core component, each tube having a second state dimensioned smaller than the first state such that when the plurality of tubes undergo a deformation from the first state to the second state each tube places a hoop stress on the core component.

2. The spinal rod of claim 1, wherein the plurality of nested tubes are deformable by applying heat.

3. The spinal rod of claim 1, wherein the plurality of nested tubes are deformed at the same time.

4. The spinal rod of claim 1, wherein a tube of the plurality of nested tubes has a different modulus of elasticity than another tube of the plurality of nested tubes.

5. The spinal rod of claim 1, wherein a tube of the plurality of nested tubes is deformed prior to deforming another of the plurality of nested tubes.

6. The spinal rod of claim 1, wherein the plurality of nested tubes are formed from a polymeric material.

7. The spinal rod of claim 6, wherein the plurality of nested tubes are formed from a PEEK material.

8. The spinal rod of claim 6, wherein the plurality of nested tubes are formed from a resorbable polymeric material.

9. The spinal rod of claim 1, wherein the core component is a metal formed from titanium, a titanium alloy, cobalt chrome, or a stainless steel alloy.

10. The spinal rod of claim 9, wherein each tube is a polymeric material having a different modulus of elasticity than the metal core component.

11. The spinal rod of claim 1, wherein each tube in the plurality of nested tubes has a thickness between 0.1 mm and 1 mm.

12. The spinal rod of claim 11, wherein each tube in the plurality of nested tubes has a thickness between 0.25 mm and 0.75 mm.

13. A method of forming a composite rod, comprising the steps of:
nesting a plurality of tubes over a core; and
applying a deforming force to the plurality of tubes such that each tube places a hoop stress on the core.

14. The method of claim 13, wherein the deforming force is heat.

15. The method of claim 13, wherein the second state inner diameter is generally equal to the outer diameter of the core.

16. The method of claim 13, wherein the nesting step and the applying step occur at the same time.

17. The method of claim 13, wherein the applying the deforming force occurs after the nesting occurs.

18. A spinal rod comprising:
a core component having a radius; and
a tube having a first state with a first state tube inner radius and a first state tube outer radius, the first state tube inner radius being greater than the core component radius,
the tube having a second state with a second state tube inner radius and a second state tube outer radius, the second state tube inner radius being generally equal to the core component radius, the tube being deformable from the first state to the second state,
wherein the core component and the tube together have an average modulus of elasticity at least 10% less than the modulus of elasticity of the core component.

19. The spinal rod of claim 18, wherein the tube has a thickness between 0.25 mm and 0.75 mM.

20. The spinal rod of claim 18 further comprising an adhesive between the core component and the tube.
